(19) United States
(12) Patent Application Publication
(10) Pub. No.: US 2011/0294109 A1
(43) Pub. Date: Dec. 1, 2011

(54) METHODS FOR DETECTING AN ANALYTE

(76) Inventors: Barbara R. Kinnaird, Richmond (CA); Lynn P. Cloney, Vancouver (CA); Paul C. Harris, Bothell, WA (US); Patrick A. Mach, Shorewood, MN (US); Mara S. Reif-Wenner, Red Wing, MN (US)

(21) Appl. No.: 12/812,812

(22) PCT Filed: Jan. 16, 2009
(86) PCT No.: PCT/US09/31260

§ 371 Date: Aug. 23, 2011

Related U.S. Application Data
(60) Provisional application No. 61/011,592, filed on Jan. 18, 2008.

(51) Int. Cl. C12Q 1/70; C12Q 1/68; G01N 33/577; G01N 33/566; G01N 33/53; G01N 33/569

(52) U.S. Cl. 435/5; 435/7.92; 435/7.33; 435/7.5; 435/6.15

(57) ABSTRACT

Methods of decreasing non-specific binding in solid phase assays for an analyte are disclosed. In the methods, the solid phase apparatus (lateral flow solid phase apparatus or capillary flow solid phase apparatus) is subjected to elevated heat. The elevated heat can be applied subsequent to application of a test sample to the solid phase apparatus.

### BACKGROUND OF THE INVENTION

Quantitative analysis of analytes in fluid samples, particularly bodily fluid samples, often provides critical information (e.g., diagnostic and treatment information for physicians and patients). In solid phase lateral flow and capillary flow assays, certain reagents are attached to a solid surface, facilitating separation of analytes. The solid phase is exposed to a sample containing the analyte; the extent of this binding is quantitated to provide a measure of the analyte concentration in the sample. Transduction of the binding event into a measurable signal, however, is affected by a number of limitations, including constraints of particle movement on the solid phase, which affect the specificity and applicability of quantitative assays. In addition, related analytes may compete with one another in an assay, rendering it difficult to assess correctly the presence of an analyte of interest.

The emergence of bacteria having resistance to commonly used antibiotics is an increasing problem with serious implications for the treatment of infected individuals. Accordingly, it is of increasing importance to determine the presence of such bacteria at an early stage and in a relatively rapid manner to gain better control over such bacteria. This also applies to a variety of other microbes.

One such microbe of significant interest is Staphylococcus aureus (“S. aureus”). This is a pathogen causing a wide spectrum of infections including: superficial lesions such as small skin abscesses and wound infections; systemic and life threatening conditions such as endocarditis, pneumonia and septicemia; as well as toxinoses such as food poisoning and toxic shock syndrome. Some strains (e.g., Methicillin-Resistant S. aureus) are resistant to all but a few select antibiotics.

Current techniques for the detection of microbes, particularly bacteria resistant to antibiotics, are generally time consuming and typically involve culturing the bacteria in pure form. Although methods of detecting analytes characteristic of S. aureus, as well as other analytes, have been described in the art, there would be advantages in improved methods of detection.

### SUMMARY

The present invention provides a method of analyzing a sample for a target analyte. Generally, the method includes providing a sample suspected of including a target analyte, providing a solid phase apparatus comprising an application point and a sample capture zone including a sample capture reagent, providing at least one labeled analyte binding agent having specificity for the target analyte, providing contact between the sample and the, sample capture zone, wherein the sample capture reagent possesses specificity for the target analyte, incubating the solid phase apparatus at a temperature greater than about 25° C. and less than about 45° C.; and detecting label in the sample capture zone.

In one aspect, the method can comprise providing a fluid sample suspected of including a target analyte, providing at least one labeled analyte binding agent having specificity for the target analyte, and providing a solid phase apparatus. The solid phase apparatus can comprise a sample application point and a sample capture zone including at least one sample capture reagent having specificity for the target analyte. The method further can comprise providing contact between the sample and the labeled analyte binding agent whereby the target analyte, if present in the sample, becomes a labeled analyte. The method further can comprise applying the sample to the application point whereby the sample moves by capillary action through the solid phase apparatus to and through the sample capture zone. The method further can comprise incubating the solid phase apparatus at a temperature greater than about 25° C. and less than about 45° C. and detecting labeled analyte in the sample capture zone, wherein detecting labeled analyte indicates the presence of the target analyte in the sample.

In some embodiments, the sample application point comprises the labeled analyte binding agent. In some embodiments, the labeled analyte binding agent comprises a particle. In some embodiments, the particle is a labeled particle. In some embodiments, the particle is coated with at least one type of analyte binding agent.

In some embodiments, the solid phase apparatus is a lateral flow solid phase apparatus. In some embodiments, the solid phase apparatus is a capillary flow solid phase apparatus.

In some embodiments, incubating the solid phase apparatus comprises incubating the solid phase apparatus at a temperature greater than about 29° C. In some embodiments, incubating the solid phase apparatus comprises incubating the solid phase apparatus at a temperature greater than about 37° C. In some embodiments, incubating the solid phase apparatus comprises incubating the solid phase apparatus at a temperature less than about 42° C. In some embodiments, the temperature varies during incubation by no more than ±1° C. In some embodiments, the temperature varies during incubation by no more than ±0.5° C. In some embodiments, the temperature varies during incubation by no more than ±0.1° C.

In some embodiments, the sample capture zone comprises two or more immobilized antibodies. In some embodiments, the analyte binding agent comprises two or more antibodies.

In some embodiments, detecting labeled analyte binding agent comprises detecting a fluorophore. In some embodiments, detecting a fluorophore comprises detecting a fluorophore visually without an instrument.

In another aspect, the present invention is drawn to methods for decreasing non-specific binding of staph detection particles, on a solid phase apparatus in an assay for Staphylococcus aureus. In the methods, a solid phase apparatus (a lateral flow solid phase apparatus or a capillary flow solid phase apparatus) and staph detection particles are used. The staph detection particles are coated with an agent that binds to Staphylococcus aureus (e.g., antibody to Staphylococcus aureus; fibrinogen). The solid phase apparatus comprises an application point and at least one sample capture zone having a sample capture reagent immobilized thereon; the sample capture reagent comprises an agent that specifically binds to Staphylococcus aureus (e.g., antibody to Staphylococcus aureus).

In certain embodiments, the staph detection particles are positioned at the application point of the solid phase apparatus; in other embodiments, the test sample comprises the staph detection particles. In either embodiment, the test sample is applied to the solid phase apparatus, and the solid phase apparatus is then subjected to elevated temperatures. Elevated temperature can be about 5 to 20 degrees Celsius above ambient temperature, and is preferably between about 35 and 40 degrees Celsius, inclusive.

Application of the test sample to the solid phase apparatus results in movement of the staph detection particles through the solid phase apparatus by capillary action. The staph detection particles move to and through the sample capture zone, and may bind to the sample capture reagent. Subjecting the solid phase apparatus to elevated temperature results in a decrease of non-specific binding of staph detection particles in the sample capture zone of the solid phase apparatus. The methods thereby enhance the sensitivity and specificity of the assays for Staphylococcus aureus.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a bar graph depicting enhanced ratios of detection particles in a sample capture zone of the solid phase apparatus, at elevated temperature compared to ambient temperature and to intermediate temperature, which are indicative of a significant decrease of non-specific binding of staph detection particles on the solid phase apparatus.

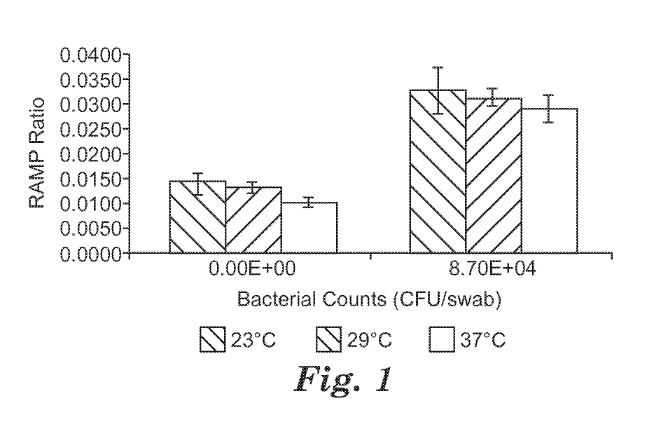

### DETAILED DESCRIPTION OF ILLUSTRATIVE EMBODIMENTS

A description of example embodiments of the invention follows. The teachings of all patents, published applications and references cited herein are incorporated by reference in their entirety. Various modifications and alterations of this invention will become apparent to those skilled in the art without departing from the scope and spirit of this invention, and it should be understood that this invention is not to be unduly limited to the illustrative embodiments set forth herein.

It has been surprisingly found that the performance of a detection assay can be improved by including a step in which the target analyte and capture reagent to which the target analyte is bound are heated prior to detecting bound target analyte. The heating step can reduce false positive results without reducing true positive results. Thus, the invention provides detection methods that can possess lower background signal than similar detection methods without the heating step. As a consequence, the present invention can provide a detection assay with improved sensitivity—i.e., capable of detecting lower concentrations of target analyte. Moreover, heating to a desired, predetermined temperature can also eliminate the inherent variability that follows from performing an incubation step of a detection assay at room temperature, which can vary from about 20° C. to about 27° C.

The terms “analyte” and “antigen” are used interchangeably and refer to various molecules (e.g., Protein A) or epitopes of molecules, or whole cells of the microorganism, that are characteristic of a microorganism (i.e., microbe) of interest. These include components of cell walls (e.g., cell wall proteins such as Protein A), external cell components (e.g., capsular polysaccharides and cell wall carbohydrates), internal cell components (e.g., cytoplasmic membrane proteins, polypeptides, polynucleotides), etc.

The term “antibody” is generally intended to include whole antibodies of any isotype (IgG, IgA, IgM, IgE, etc.), and fragments thereof from vertebrate (e.g., mammalian) species that are specifically reactive with non-self materials, e.g., proteins.

The term “mucus-containing sample” refers to a sample that includes, or is derived from, mucosal membranes and/or mucosal tissues, which are used interchangeably and refer to the surfaces of the nasal (including anterior nares, nasopharyngeal cavity, etc.), oral (e.g., mouth), outer ear, middle ear, vaginal cavities, and other similar tissues. Examples include mucosal membranes such as buccal, gingival, nasal, ocular, tracheal, bronchial, gastrointestinal, rectal, urethral, ureteral, vaginal, cervical, and uterine mucosal membranes.

“Specificity” and variations thereof refers to the quality of being able to differentially select between alternatives, without specific regard to the mechanism of the selection. That is, specificity may be based on, for example, size, immunoreactivity, etc.

“Whole cell” means a biologically active prokaryotic or eukaryotic cell that retains its structure during separation from other biological materials, but does not necessarily need to be able to reproduce.

The terms “comprises” and variations thereof do not have a limiting meaning where these terms appear in the description and claims.

As used herein, “a,” “an,” “the,” “at least one,” and “one or more” are used interchangeably. Thus, for example, an analyte-binding material that comprises “an” antibody can be interpreted to mean that the analyte-binding material includes “one or more” antibodies that bind different analytes.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The invention generally relates to methods of performing detection assays in which a target analyte is permitted to bind with (a) a labeled analyte binding agent that possesses specificity for the target analyte, and (b) a sample capture zone that includes at least one capture reagent that possesses specificity for the target analyte. The assays are performed in a solid phase apparatus as described herein. The presence of the target analyte in the sample is indicated by detecting label above background in the sample capture zone. It has been found that incubating the solid phase apparatus and, in particular, the sample capture zone of the solid phase apparatus, after contacting the sample capture zone with the target analyte can improve the performance of such assays.

Analytes (e.g., proteins, nucleic acids, cell walls, or microorganisms) in the sample are detected using materials that possess some degree of specificity for the target analyte. These materials include capture reagents and labeled analyte binding agents. The capture reagents capture the target analyte in an area (e.g., a sample capture zone) where the analyte can be detected. The labeled analyte binding agent includes a label that allows the target analyte to be detected if captured by a capture reagent. Generally, the method includes heating the sample capture zone while the target analyte moves to and through the sample capture zone and before the bound target analyte is detected. Generally, unless otherwise indicated for a particular embodiment, the sample capture zone may be heated prior to, simultaneously with, or subsequent to, contacting the target analyte with the labeled analyte binding agent.

### Analyte Binding Agents:

The present disclosure includes methods that include analyte binding agents, as described herein. Analyte binding agents may be used as capture reagents. The methods of the invention utilize analyte binding agents that are members of a specific binding pair, in which a first member of the binding pair (the analyte) reacts specifically with a second member (e.g., an analyte binding agent). Specific interaction between the members of the binding pair indicates that the first member of the binding pair preferentially binds or otherwise interacts with the second member of the binding pair, preferably to the exclusion of any binding to another compound in the assay. In some embodiments, the second member of the binding pair can be an antibody (e.g., an antibody to an antigenic component of the analyte). In this embodiment, the assay is an immunoassay. In other embodiments, the second member of the binding pair can be a receptor (e.g., a protein receptor or a hormone receptor) or a protein molecule that selectively binds to other molecules (e.g., a protein molecule such as fibrinogen, which selectively binds to another molecule such as Clumping Factor).

In some embodiments, the analyte binding agent and/or the capture reagent may include two or more materials having specificity for the target analyte. In such embodiments, the analyte binding agent and/or capture reagent can include materials (e.g., two or more antibodies) that have specificity for different recognition sites (e.g., different molecules characteristic of the microorganism, different epitopes of an antigen, etc.) of the analyte. In other cases, the analyte binding agent and/or capture reagent can include materials that have specificity for repeating recognition sites of the analyte (e.g., a protein present in multiple copies in the microorganism, an epitope present in multiple sites within an antigen, etc.).

In some embodiments, the analyte binding agent and/or the capture reagent can include one or more antibodies that have specificity for the target analyte. For example, the analyte binding agent and/or the capture reagent can include one or more S. aureus antibodies. “S. aureus antibody” refers to an immunoglobulin having the capacity to specifically bind a S. aureus antigen, and includes antibody fragments capable of specifically binding a S. aureus antigen.

The antibodies can be monoclonal, polyclonal, or combinations thereof. Antibodies can be fragmented using conventional techniques and the fragments screened for utility in the same manner as whole antibodies. Thus, the antibodies can include segments of proteolytically cleaved or recombinantly prepared portions of an antibody molecule that are capable of selectively reacting with a certain protein. Non-limiting examples of such proteolytic and/or recombinant fragments include Fab, F(ab′)₂, Fv, and single chain antibodies (scFv) containing a VL and/or VH domain joined by a peptide linker. The scFv's can be covalently or non-covalently linked to form antibodies having two or more sites that can specifically bind to a target analyte.

Various S. aureus antibodies are known in the art. For example, S. aureus antibodies are commercially available from Sigma-Aldrich and Accurate Chemical. Further, S. aureus antibodies are described in U.S. Pat. No. 4,902,616, including the monoclonal antibody referred to as MAb 12-9 antibody, available from Inhibitex, Atlanta, Ga. In certain embodiments, an antibody is selected from those described herein (e.g., selected from the group consisting of MAb-76, MAb-107, affinity-purified RxClf40, affinity-purified GxClf40, MAb 12-9), fragments thereof, and combinations thereof. Such antibodies are also disclosed in U.S. Patent Application Publication No. 2008-0118937-A1, U.S. patent application Ser. No. 11/562,747, and U.S. Provisional Patent Application Ser. No. 60/867,089.

In some embodiments, the antibodies may be monoclonal antibodies. In particular embodiments, the antibodies may be monoclonal antibodies that bind to Protein A of Staphylococcus aureus (also referred to herein as “S. aureus” or “Staph A”).

More particularly, in one embodiment suitable monoclonal antibodies, and antigen binding fragments thereof, are those that demonstrate immunological binding characteristics of monoclonal antibody 76 as produced by hybridoma cell line 358A76.1. Murine monoclonal antibody 76 is a murine IgG2A, kappa antibody isolated from a mouse immunized with Protein A. In accordance with the Budapest Treaty, hybridoma 358A76.1, which produces monoclonal antibody 76, was deposited on Oct. 18, 2006 in the American Type Culture Collection (ATCC) Depository, 10801 University Boulevard, Manassas, Va. 20110-2209, and was given Patent Deposit Designation PTA-7938 (also referred to herein as accession number PTA-7938). The hybridoma 358A76.1 produces an antibody referred to herein as “MAb-76.” MAb-76 is also referred to herein as “Mab76,” “Mab-76,” “MAb 76,” “monoclonal 76,” “monoclonal antibody 76,” “76,” “M76,” or “M 76,” and all are used interchangeably herein to refer to immunoglobulin produced by hybridoma cell line 358A76.1 as deposited with the American Type Culture Collection (ATCC) on Oct. 18, 2006, and assigned Accession No. PTA-7938.

In another embodiment, suitable monoclonal antibodies, and antigen binding fragments thereof, are those that demonstrate immunological binding characteristics of monoclonal antibody 107 as produced by hybridoma cell line 358A107.2. Murine monoclonal antibody 107 is a murine IgG2A, kappa antibody isolated from a mouse immunized with Protein A. In accordance with the Budapest Treaty, hybridoma 358A107.2, which produces monoclonal antibody 107, was deposited on Oct. 18, 2006 in the American Type Culture Collection (ATCC) Depository, 10801 University Boulevard, Manassas, Va. 20110-2209, and was given Patent Deposit Designation PTA-7937 (also referred to herein as accession number PTA-7937). The hybridoma 358A107.2 produces an antibody referred to herein as “MAb-107.” MAb-107 is also referred to herein as “Mab107,” “Mab-107,” “MAb 107,” “monoclonal 107,” “monoclonal antibody 107,” “107,” “M107,” or “M 107,” and all are used interchangeably herein to refer to immunoglobulin produced by the hybridoma cell line as deposited with the American Type Culture Collection (ATCC) on Oct. 18, 2006, and given Accession No. PTA-7937.

In some embodiments, suitable monoclonal antibodies are also those that inhibit the binding of monoclonal antibody MAb-76 to Protein A of S. aureus. The present invention includes monoclonal antibodies that bind to the same epitope of Protein A of S. aureus that is recognized by monoclonal antibody MAb-76. Methods for determining if a monoclonal antibody inhibits the binding of monoclonal antibody MAb76 to Protein A of S. aureus and determining if a monoclonal antibody binds to the same epitope of Protein A of S. aureus that is recognized by monoclonal antibody MAb-76 are well known to those skilled in the art of immunology.

In some embodiments, suitable monoclonal antibodies are also those that inhibit the binding of monoclonal antibody MAb-107 to Protein A of S. aureus. The present invention includes monoclonal antibodies that bind to the same epitope of Protein A of S. aureus that is recognized by monoclonal antibody MAb-107. Methods for determining if a monoclonal antibody inhibits the binding of monoclonal antibody MAb-107 to Protein A of S. aureus and determining if a monoclonal antibody binds to the same epitope of Protein A of S. aureus that is recognized by monoclonal antibody MAb-107 are well known to those skilled in the art of immunology.

Also included in the present invention include various antibody fragments, also referred to as antigen binding fragments. Examples of antibody fragments include, for example, Fab, Fab′, Fd, Fd′, Fv, dAB, and F(ab′)₂ fragments produced by proteolytic digestion and/or reducing disulfide bridges and fragments produced from an Fab expression library. Such antibody fragments can be generated by techniques well known in the art.

Monoclonal antibodies useful in the present invention include, but are not limited to, humanized antibodies, chimeric antibodies, single chain antibodies, single-chain Fvs (scFv), disulfide-linked Fvs (sdFv), Fab fragments, F(ab′) fragments, F(ab′)₂ fragments, Fv fragments, diabodies, linear antibodies fragments produced by a Fab expression library, fragments including either a VL or VH domain, intracellularly-made antibodies (i.e., intrabodies), and antigen-binding antibody fragments thereof.

Monoclonal antibodies useful in the present invention may be of any isotype. The monoclonal antibodies useful in the present invention may be, for example, murine IgM, IgG1, IgG2a, IgG2b, IgG3, IgA, IgD, or IgE. The monoclonal antibodies useful in the present invention may be, for example, human IgM, IgG1, IgG2, IgG3, IgG4, IgA1, IgA2, IgD, or IgE. In some embodiments, the monoclonal antibody may be murine IgG2a, IgG1, or IgG3. With the present invention, a given heavy chain may be paired with a light chain of either the kappa or the lambda form.

Monoclonal antibodies useful in the present invention can be produced by an animal (including, but not limited to, human, mouse, rat, rabbit, hamster, goat, horse, chicken, or turkey), chemically synthesized, or recombinantly expressed. Monoclonal antibodies useful in the present invention can be purified by any method known in the art for purification of an immunoglobulin molecule, for example, by chromatography (e.g., ion exchange, affinity, and sizing column chromatography), centrifugation, differential solubility, or by any other standard technique for the purification of proteins.

In some embodiments, suitable antibodies also include a high avidity anti-Staphylococcus aureus Clumping Factor protein polyclonal antibody composition that detects recombinant Clumping Factor (rClf40) protein of S. aureus at a concentration of at least 1 picogram per milliliter (pg/mL) such as, for example, up to 100 pg/mL. Suitable antibodies also include a high avidity anti-Staphylococcus aureus Clumping Factor protein polyclonal antibody composition demonstrating at least a 4-fold increase in detection sensitivity in comparison to a Staphylococcus aureus clumping Factor Protein antiserum.

In certain embodiments, a high avidity anti-Staphylococcus aureus Clumping Factor protein polyclonal antibody composition may be prepared by a method that includes obtaining antiserum from an animal immunized with recombinant Clumping Factor (rClf40) protein of S. aureus; binding the antiserum to a S. aureus Clumping Factor (Clf40) protein affinity column; washing the column with a wash buffer having 0.5 M salt and a pH of 4; and eluting the high avidity anti-S. aureus Clumping Factor protein polyclonal antibody composition from the column with an elution buffer with a pH of 2. Herein, the high avidity anti-Staphylococcus aureus clumping factor polyclonal antibody compositions from rabbits and goats are referred to as affinity-purified RxClf40 and affinity-purified GxClf40, respectively. In some embodiments, the high avidity anti-Staphylococcus aureus Clumping Factor protein polyclonal antibody composition may be obtained by a method that further includes enriching the antiserum for the IgG class of antibodies prior to binding the antiserum to a S. aureus Clumping Factor (Clf40) protein affinity column. Such enrichment may eliminate non-immunoglobulin proteins from the composition and/or enrich for the IgG class of antibodies within the sample.

As used herein, antiserum refers to the blood from an immunized host animal from which the clotting proteins and red blood cells (RBCs) have been removed. An antiserum to a target antigen may be obtained by immunizing any of a variety of host animals. Any of a wide variety of immunization protocols may be used.

Antibody avidity is a measure of the functional affinity of a composition of polyclonal antibodies. Avidity is the compound affinity of multiple antibody/antigen interactions. That is, avidity is the apparent affinity of antigen/antibody binding, not the true affinity. Despite the heterogeneity of affinities in most antisera, one can characterize such populations by defining an average affinity (K₀).

In certain embodiments of the present invention, the two or more labeled antibodies include one monoclonal antibody, such as MAb-107, and one polyclonal antibody composition, such as affinity-purified RxClf40.

In many embodiments, the analyte binding agent and/or capture reagent can include antibodies. In some embodiments, antibodies can be attached to particulate material, a membrane, or other solid support material. Antibodies can be attached to a solid support material through either covalent attachment or non-covalent attachment.

For example, suitable methods of attaching antibodies to a solid support include that disclosed in U.S. Patent Application Publication No. 2003/0162236 using cyanoborohydride chemistry in skim milk.

Non-covalent attachment of an antibody to a solid support material includes attachment by passive adsorption and/or absorption, ionic interaction, or hydrogen bonding, for example.

Representative methods for covalent attaching an antibody to a particulate support material include chemical crosslinkers, such as heterobifunctional crosslinking compounds (i.e., “linkers”) that react to form a bond between reactive groups (such as hydroxyl, amino, amido, or sulfhydryl groups) in an antibody and other reactive groups (of a similar nature) in the support material. This bond may be, for example, a peptide bond, disulfide bond, thioester bond, amide bond, thioether bond, and the like.

Antibodies may be covalently bonded to a particulate support material by any of the methods known in the art. For example, glutaraldehyde, aldehyde-Schiffs base, n-hydroxyl succinimide, azlactone, cyanogen bromide, maleic anhydride, etc., may be used as attachment chemistries.

One example of a non-covalent labeling of antibodies included in the present invention is the well-known biotin-avidin system. Such a system can also be used in the labeling system of the antibodies for detection, particularly when enzymatic detection systems are used as in an ELISA.

Avidin-biotin affinity-based technology has found wide applicability in numerous fields of biology and biotechnology. The affinity constant between avidin and biotin is remarkably high (the dissociation constant, Kd, is approximately 10⁻¹⁵ M, see, Green, Biochem. J., 89, 599 (1963)) and is not significantly lessened when biotin is coupled to a wide variety of biomolecules. Numerous chemistries have been identified for coupling biomolecules to biotin with minimal or negligible loss in the activity or other desired characteristics of the biomolecule. A review of the biotin-avidin technology can be found in Applications of Avidin-Biotin Technology to Affinity-Based Separation, Bayer, et al., J. of Chromatography, pgs. 3-11 (1990).

Streptavidin, and its functional homolog avidin, are tetrameric proteins, having four identical subunits. Streptavidin is secreted by the actinobacterium, Streptomyces avidinii. A monomer of streptavidin or avidin contains one high-affinity capture reagent for the water-soluble vitamin biotin and a streptavidin or avidin tetramer binds four biotin molecules.

Biotin, also known as vitamin H or cis-hexahydro-2-oxo-1H-thieno-[3-,4]-imidazole-4-pentanoic acid, is a basic vitamin which is essential for most organisms including bacteria and yeast. Biotin has a molecular weight of 244 daltons, much lower than its binding partners, avidin and streptavidin. Biotin is also an enzyme cofactor of pyruvate carboxylase, trans-carboxylase, acetyl-CoA-carboxylase and beta-methylcrotonyl-CoA carboxylase which together carboxylate a wide variety of substrates.

Both streptavidin and avidin exhibit extremely tight and highly specific binding to biotin which is one of the strongest known non-covalent interactions between proteins and ligands, with a molar dissociation constant of 10⁻¹⁵ molar (M) (Green, Advances in Protein Chemistry, Vol. 29, pp. 85-133 (1975)), and a t1/2 of ligand dissociation of 89 days (Green, N. Mex., Advances in Protein Chemistry, Vol. 29, pp. 85-133 (1975)). The avidin-biotin bond is stable in serum and in the circulation (Wei et al., Experientia, Vol. 27, pp. 366-368 (1970)). Once formed, the avidin-biotin complex is unaffected by most extremes of pH, organic solvents and denaturing conditions. Separation of streptavidin from biotin requires conditions, such as 8M guanidine, pH 1.5, or autoclaving at 121° C. for 10 minutes (min).

Antibodies may be biotinylated using any known methodologies. For example, antibodies may be biotinylated chemically, using activated biotin analogues, such as N-hydroxysuccinimidobiotin (NHS-biotin), which is commercially available from Pierce Chemical Company, Rockford, Ill., and requires the presence of a free primary amino group on the antibody.

In certain embodiments, a capture reagent may include a material that possesses specificity for a target analyte/labeled analyte binding agent complex that is based on a characteristic other than immunoreactivity. For example, certain materials may possess specificity for a target analyte/labeled analyte binding agent complex based on the size of the complex versus the size of the individual components (i.e., unbound target analyte and unbound labeled analyte binding agent). Such capture reagents can include any suitable size-selective material known to those skilled in the art such as, for example, particulate materials, membranes, gels (e.g., agarose), or size exclusion material.

An analyte binding agent and/or capture reagent can include a solid support material. Solid support materials can include particulate materials, membranes, gels, or other solid support materials such as the surfaces of capillary tubes or microchannels. Exemplary solid supports can include materials such as nitrocellulose, polystyrene, polypropylene, nylon, gold sols, and/or latex particles, and the like. For certain embodiments, the solid support may be a particulate material or membrane. For certain embodiments, the solid support material may be a particulate material (e.g., polystyrene and/or latex beads having an average particle size of less than 1 micron such as, for example, approximately 0.3 micron).

An analyte binding agent and/or capture reagent can include a solid support material having one or more antibodies disposed on the solid support. In certain embodiments, the solid support material (e.g., each particle of the particulate material) can include at least two antibodies that bind different target analytes.

For example, in certain embodiments, the analyte binding agent and/or capture reagent includes a solid support material having antibodies MAb-76 and affinity-purified RxClf40 disposed thereon, and a label. The MAb-76 and RxClf40 may be provided in a ratio of, for example 1:1, 2:1, 1:2, 3:1, or 1:3.

In other embodiments, an analyte binding agent and/or capture reagent can include a solid support material having antibodies MAb-76 and MAb-12-9 disposed thereon. In some embodiments, the antibodies may be present in a concentration combination of, for example, 1 μg/mL MAb-76 and 7.5 μg/mL MAb 12-9). Depending on the desired results and antibodies used, the concentrations and densities of immobilized antibodies can be varied by one of skill in the art.

### Samples and Target Analytes:

A sample can be obtained from any suitable source. In some embodiments, the test sample can be a clinical sample such as, for example, a physiological fluid, e.g., blood, saliva, ocular lens fluid, synovial fluid, cerebral spinal fluid, pus, sweat, exudate, urine, mucus, lactation milk, or the like. Further, the test sample may be derived from a body site, e.g., wound, skin, nares, scalp, nails, etc.

In some embodiments, the sample may be a mucus-containing sample. Mucus-containing samples may include, for example, nasal samples (from, e.g., anterior nares, nasopharyngeal cavity, nasal cavities, anterior nasal vestibule, etc.), as well as samples from the outer ear, middle ear, mouth, rectum, vagina, or other similar tissue. Examples of specific mucosal tissues include buccal, gingival, nasal, ocular, tracheal, bronchial, gastrointestinal, rectal, urethral, ureteral, vaginal, cervical, and uterine mucosal membranes.

Besides physiological fluids, other suitable sources of test samples may include other liquids as well as solid(s) dissolved in a liquid medium. Samples of interest may include process streams, water, soil, plants or other vegetation, air, surfaces (e.g., contaminated surfaces), and the like. Samples can also include cultured cells.

In some embodiments, the test sample can be a food or beverage sample such as, for example, a food or beverage raw material, an in-process sample, or a finished-product sample. In some embodiments, the test sample can be an environmental sample such as, for example, a surface residue sample or a surface water sample.

The art describes various patient sampling techniques for the detection of analytes (e.g., microbes, such as S. aureus or analytes therefrom). Such sampling techniques are suitable for the methods of the present invention as well.

Methods of the invention may be used to detect the presence of certain microorganisms in a sample. In certain embodiments, the microorganism can include a prokaryotic or eukaryotic organism such as, for example, Gram positive bacteria, Gram negative bacteria, fungi, protozoa, mycoplasma, yeast, viruses, and even lipid-enveloped viruses. In particular embodiments, the microorganism can be a member of the family Enterobacteriaceae, or the family Micrococcaceae or the genera Staphylococcus spp., Streptococcus spp., Pseudomonas spp., Enterococcus spp., Salmonella spp., Legionella spp., Shigella spp. Yersinia spp., Enterobacter spp., Escherichia spp., Bacillus spp., Listeria spp., Vibrio spp., Corynebacteria spp., as well as herpes virus, Aspergillus spp., Fusarium spp., and Candida spp. In specific embodiments, the method may be used to detect particularly virulent organisms such as, for example, Staphylococcus aureus (including resistant strains such as Methicillin Resistant Staphylococcus aureus (MRSA)), S. epidermidis, Streptococcus pneumoniae, S. agalactiae, S. pyogenes, Enterococcus faecalis, Vancomycin Resistant Enterococcus (VRE), Vancomycin Resistant Staphylococcus aureus (VRSA), Vancomycin Intermediate-resistant Staphylococcus aureus (VISA), Bacillus anthracis, Pseudomonas aeruginosa, Escherichia coli, Aspergillus niger, A. fumigatus, A. clavatus, Fusarium solani, F. oxysporum, F. chlamydosporum, Listeria monocytogenes, Listeria ivanovii, Vibrio cholera, V. parahemolyticus, Salmonella cholerasuis, S. typhi, S. typhimurium, Candida albicans, C. glabrata, C. krusei, Enterobacter sakazakii, E. coli O157, and multiple drug resistant Gram negative rods (MDR).

Thus, the methods may be used to detect Gram positive and/or Gram negative bacteria. In certain embodiments, the methods may be used to detect Gram positive bacteria such as, for example, Staphylococcus aureus. Typically, Gram positive bacteria can be detected by detecting the presence of a cell wall component characteristic of the bacteria, such as a cell wall protein. Also, of particular interest are antibiotic resistant microbes including MRSA, VRSA, VISA, VRE, and MDR. Typically, these can be detected by additionally detecting the presence of an internal cell component, such as a membrane protein, transport protein, enzyme, etc., responsible for antibiotic resistance.

Other microorganisms of interest can include viruses such as, for example, those belonging to the classification groups including Adenoviridae, Hepadnaviridae, Herpesviridae, Papovaviridae, Parvoviridae, Poxyiridae, Arenaviridae, Astroviridae, Bunyaviridae, Caliciviridae, Coronaviridae, Filoviridae, Flaviviridae, Orthomyxoviridae, Paramyxoviridae, Picornaviridae, Reoviridae, Retroviridae, Rhabdoviridae, and Togaviridae. In certain embodiments, the methods can be used to detect the presence of influenza virus in a sample. Thus, the methods can be used to detect, for example, type A influenza virus and/or type B influenza virus.

The target analyte can include, for example, a cell wall protein such as Protein A or microbial surface components recognizing adhesive matrix molecules (MSCRAMMs) such as fibrinogen-binding proteins (e.g., Clumping Factor), fibronectin-binding proteins, collagen-binding proteins, heparin/heparin-related polysaccharides binding proteins, and the like. Protein A and Clumping Factor, such as fibrinogen-binding proteins and clumping factors A, B, and Efb, may be particularly useful for detecting Staphylococcus aureus. Other external cell components that may be useful as a target analyte include, for example, capsular polysaccharides and cell wall carbohydrates (e.g., teichoic acid and lipoteichoic acid).

A target analyte may alternatively include an internal cell component, which may or may not be associated with a cell membrane. Internal cell components may be particularly useful in analyzing antibiotic resistant microbes, such as MRSA, VRSA, VISA, VRE, and MDR. Internal cell components that can be characteristic of such microbes include membrane proteins. Examples of such membrane proteins include cytoplasmic membrane proteins, inner membrane proteins, outer membrane proteins, and cell membrane proteins. Cytoplasmic membrane proteins, such as penicillin binding proteins (PBP) (e.g., PBP2′ or PBP2a) can be particularly characteristic of antibiotic resistant microbes. For example, the cytoplasmic membrane protein PBP2′ is characteristic of MRSA.

In some embodiments, the method may be designed to include two or more target analytes of the microorganism. Thus, the method may include, as target analytes, one or more cell wall proteins, one or more alternative external cell components, one or more internal component, or any combination thereof.

If desired, the test sample can be diluted or otherwise treated or prepared; for example, it can be diluted with a solution (e.g., an aqueous solution or buffer) or the sample can be subjected to a physical (e.g., filtration, homogenation, or sonication) or chemical treatment (e.g., pH adjustment, mucolytic treatment).

A sample can be prepared using a wide variety of means well-known to those of skill in the art. For example, in some embodiments, the target analyte may be a whole cell. In such cases, the sample may require minimal preparation. In other embodiments, the target analyte may be present on cell fragments or in certain fractions of cells. In such embodiments, whole cells may be physically, chemically, or otherwise disrupted to make available for analysis a target analyte characteristic of the microorganism that is a component (e.g., cell wall component, plasma membrane component, intracellular membrane component, cytoplasm component, etc.) of the microorganism.

Physical means of disrupting a cell include, for example, sonication, boiling or other heating means, and vortexing with glass beads. Alternatively, the sample could be disrupted using various chemical reagents, which can include one or more components.

In certain embodiments, methods of the present invention include lysing the cells in the test sample. Lysing cells in the sample can include contacting the cells with a lysing agent or physically lysing the cells. Lysing can be conducted under conventional conditions, such as, for example, at a temperature of 5° C. to 37° C. (although perhaps as high as 50° C.). In some embodiments, lysing may be performed at a temperature of, for example, from 15° C. to 25° C. The sample subjected to lysing can contain uncultured cells, i.e., a direct test sample, although cultured cells can be used as well. Lysing can occur upon physically lysing the cells. Physical lysing can occur upon vortexing the test sample with glass beads, sonicating, heating and boiling, or subjecting the test sample to high pressure, such as occurs upon using a French press.

Lysing can also occur using a lysing agent. Suitable lysing agents include, for example, enzymes (e.g., protease, glycosidases, nucleases). Exemplary enzymes include lysostaphin, pepsin, glucosidase, galactosidase, lysozyme, achromopeptidase, endopeptidases, N-acetylmuramyl-L-alanine amidase, endo-beta-N-acethylglucosaminidase, ALE-1, DNase, and RNase. Various combinations of enzymes can be used if desired. Lysostaphin is particularly useful in methods of detecting the presence of Staphylococcus aureus. Other lysing agents include salts (e.g., chaotrophic salts), solubilizing agents (e.g., detergents), reducing agents (e.g., beta-mercaptoethanol (BME), dithiothreitol (DTT), dithioerythritol (DTE), cysteine, n-acetyl cysteine)), acids (e.g., HCl), and bases (e.g., NaOH). Such lysing agents may be more suitable for certain organisms than for others, for example, they can be more suitable for use with Gram negative bacteria than with Gram positive bacteria.

Various combinations of lysing agents and/or methods can be used if desired.

Methods of lysing are further discussed in U.S. Patent Application Publication No. 2005/0153370 A1. In particular, such lysing methods involve detecting one or more components of cell walls that are characteristic of a species of interest (e.g., a microbe of interest), and optionally one or more internal cell components that are further characteristic of a species of interest (e.g., an antibiotic resistant microbe of interest). It is believed that the cell wall fragments analyzed are solid pieces of cell wall. That is, it is believed that they are not solubilized upon lysing; rather, the cell wall is merely broken into pieces. Furthermore, the cell wall component that is analyzed is still part of (i.e., in or on) the cell wall fragments. That is, they are not solublized upon lysing. Significantly, this enhances the signal of the cell wall component relative to the same component in an unlysed cell.

In embodiments in which the sample is a mucus-containing sample, it can be further treated, either before or after lysing, with at least one reagent that can include a mucolytic agent (e.g., bacteriophage). Treatment of mucus-containing samples with mucolytic agents can reduce the interference resulting from the presence of mucus during the analysis. Examples of mucolytic agents, pretreatment methods, and sample preparation systems are described, for example, in U.S. Provisional Patent Application Ser. No. 60/867,093. As a result of the system and methods of pretreatment, samples having relatively low concentrations of the microorganism can be evaluated. Thus, advantageously, such methods can provide improved sensitivity. For example, for certain embodiments, the test sample may include a relatively low concentration of, for example, Staphylococcus aureus. Such relatively low concentrations include, for example, less than 5×10⁴ colony forming units (“cfu”) per milliliter (cfu/mL) of microbe, less than 5×10³ cfu/mL, less than 1000 cfu/mL, and even as low as 500 cfu/mL. Microorganisms, such as S. aureus, can be detected at high levels as well, ranging up to as much as 5×10⁷ cfu/mL, for example.

### Solid Phase Assays:

A description of example embodiments of the invention follows. The teachings of all patents, published applications and references cited herein are hereby incorporated by reference in their entirety.

The present invention pertains broadly to methods for decreasing non-specific binding in solid phase assays for the assessment of a target analyte in a sample. The solid phase assays of the invention can be lateral flow solid phase assays or capillary flow solid phase assays.

An assay, as used herein, refers to an in vitro procedure for analysis of a sample to determine the presence, absence, or quantity of any analyte of interest that can be detected by its selective interaction with two or more binding agents. The methods of the invention utilize analyte binding agents, as described herein. The “target analyte,” as used herein, refers to any molecule that can be detected by its selective interaction with two or more binding agents. The analyte can be a molecule with two or more epitopes; an antigen; a hapten; a biomolecule (e.g.; a protein) characteristic of a prion, a virion, a prokaryotic microorganism (e.g., a bacterium) or a eukaryotic organism (e.g., a fungal cell, an animal cell, a plant cell); or a prokaryotic or eukaryotic cell or a fragment thereof. The two or more epitopes of the target analyte may be identical epitopes or they may be nonidentical epitopes.

In the methods of the invention, a test sample is assessed. The test sample can either be a sample which is suspected of containing the target analyte, or a test sample which is suspected of not containing the target analyte.

The assays utilize “detection particles.” The detection particles are particles which can be coated with an analyte binding agent (the second member of the binding pair) for the target analyte. In a preferred embodiment, the detection particles are liposomes, colloidal gold; organic polymer latex particles, inorganic fluorescent particles or phosphorescent particles. In a particularly preferred embodiment, the particles are polystyrene latex beads, and most particularly, polystyrene latex beads that have been prepared in the absence of surfactant, such as surfactant free Superactive Uniform Aldehyde/Sulfate Latexes (Invitrogen Corp.; Carlsbad, Calif.). Generally, a single population of detection particles is used; the detection particles are coated with at least one type of analyte binding agent for the target analyte. For example; a single population of detection particles, coated with one or more types of analyte binding agent, or more than one population of detection particles, each population coated with one or more types of analyte binding agent, can be used. Representative analyte binding agents include antibodies (or fragments thereof); haptens; drug conjugates; receptors; or other binding partners.

The detection particles must be sufficiently small to be transported along a membrane or through a capillary channel by capillary action of fluid, and also sufficiently small for a complex of the target analyte and the detection particles, as described herein, to be transported by capillary action.

The particles can be labeled to facilitate detection. The particles are labeled by a means which does not significantly affect the physical properties of the particles; for example, the particles are labeled internally (that is; the label is included within the particle, such as within the liposome or inside the polystyrene latex bead). Representative labels include luminescent labels; chemiluminescent labels; phosphorescent labels; enzyme-linked labels; chemical labels, such as electroactive agents (e.g., ferrocyanide); and colorimetric labels, such as dyes or fluorescent labels. In one embodiment, a fluorescent label is used. In another embodiment, phosphorescent particles are used, particularly “up-converting” phosphorescent particles, such as those described in U.S. Pat. No. 5,043,265.

Generally, the method may be used in conjunction with any suitable analytical technique. Analytical techniques useful in methods of the present invention can be any of a wide variety of conventional techniques known to one of skill in the art. For example, such methods can include the use of fluorimetric immunochromatography (e.g., rapid analyte measurement procedure such as that described in U.S. Pat. Nos. 5,753,517, 6,509,196, and U.S. Patent Application Publication Nos. 2003/0162236 and 2003/0199004), acoustic wave sensors, enzyme-linked immunosorbent assays (ELISA, e.g., colorimetric ELISA), and other colorimetric techniques (e.g., colorimetric sensors including polydiacetylene (PDA) materials), as well as surface plasmon resonance (SPR, using biosensors of the type available from Biacore, Upsala, Sweden).

Thus, the analyte binding agent can include any tag or label suitable for use with a desired analytical technique, which can include both direct and indirect methods of detection. In some embodiments, a label can be a direct label (e.g., an enzyme such as horseradish peroxidase, glucose oxidase, alkaline phosphatase), which can be attached to the analyte binding agent through a variety of attachment chemistries known in the art, such as that disclosed in P. Tijssen Practice and Theory of Enzyme Immunoassays, Laboratory Techniques in Biochemistry and Molecular Biology, Vol. 15, Elsevier Science Publishers, B.V. New York, N.Y. (1985).

In certain embodiments, the analyte binding agent can include a solid support material (e.g., a particulate material) that includes a plurality of labels. For example, the analyte binding agent may include a particle that also includes multiple copies of an appropriate ELISA enzyme. In this way, the label generated by a labeled analyte binding agent-target analyte complex may be amplified compared to methods in which one label is provided per target analyte. As a result, certain embodiments of the assay may possess improved sensitivity—i.e., be capable of detecting lower concentrations of target analyte and, therefore, be capable of detecting lower concentrations of a microorganism in a sample.

Alternatively, the analyte binding agent can include an indirect label such as, for example, biotin, which is then used with an enzyme conjugate. Other indirect labels include, for example, haptens or other antigens that may be detected using labeled antibodies, as is known to one of skill in the art.

In an exemplary system biotin can be linked to an antibody through a polyethylene oxide linker using N-hydroxysuccininimide chemistry, for example (e.g., PEO₄—NHS). In this embodiment, the biotin is considered the indirect label and the method includes reacting the biotin-labeled antibodies with an enzyme conjugate linked to streptavidin or avidin, for example. Such biotin-avidin system is a well known labeling system of the antibodies for detection, particularly when enzymatic detection systems are used as in an ELISA. The enzyme can be a wide variety of enzymes known for use in ELISAs, including, for example, alkaline phosphatase, horseradish peroxidase, glucose oxidase, etc. such as those disclosed in P. Tijssen Practice and Theory of Enzyme Immunoassays, Laboratory Techniques in Biochemistry and Molecular Biology, Vol. 15, Elsevier Science Publishers, B.V. New York, N.Y. (1985).

The enzyme can be detected colorimetrically using an appropriate chromogenic enzyme substrate, such as para-nitrophenyl phosphate for phosphatase enzyme labels, tetramethyl benzidine or diamino benzidine for peroxidase enzymes and formazan salts as end products of glucose oxidation by glucose oxidase. In addition to colorimetric detection, alternative methods of detection can include fluorimetric detection, detection of a radiolabels, and luminescence, for example.

The assays of the invention also utilize a solid phase apparatus. In one embodiment, the solid phase apparatus is a lateral flow solid phase apparatus. In another embodiment, the solid phase apparatus is a capillary flow solid phase apparatus. In some embodiments, the target analyte can be detected using a solid phase apparatus such as, for example, an immunochromatographic device such as, for example, a lateral flow device. The immunochromatographic device can be a lateral flow device that includes a sample capture zone on a solid support material (e.g., a nitrocellulose membrane attached to Mylar backing) having antibodies (e.g., MAb 107 and affinity-purified RxClf40) disposed thereon, analogous to those devices described in U.S. Patent Application Publication No. 2003/0199004. In some embodiments, the nitrocellulose having antibodies disposed thereon is blocked against non-specific using polyvinyl alcohol.

Optionally, the immunochromatographic device can further include a control capture zone, analogous to those devices described in U.S. Patent Application Publication No. 2003/0199004.

The lateral flow solid phase apparatus can be any solid phase apparatus designed for a lateral flow assay, such as the RAMP™ apparatus (Response Biomedical, Burnaby, British Columbia, Canada; see, e.g., apparatus described in U.S. Pat. Nos. 6,509,196; 7,175,992). Generally, the lateral flow solid phase apparatus includes a membrane through which the test sample will flow. The membrane can be made of a substance having the following characteristics: sufficient porosity to allow capillary action of fluid along its surface and through its interior; the ability to allow movement of coated particles (e.g., detection particles as described herein) or complexes of particles and target analytes, by capillary action (i.e., it must not block the particles or complexes of particles and target analyte); and the ability to be wet by the fluid containing the analyte (e.g., hydrophilicity for aqueous fluids, hydrophobicity for organic solvents). Hydrophobicity of a membrane can be altered to render the membrane hydrophilic for use with aqueous fluid, by processes such as those described in U.S. Pat. No. 4,340,482 or U.S. Pat. No. 4,618,533. Nonlimiting examples of membrane substances include: cellulose, cellulose nitrate, cellulose acetate, glass fiber, nylon, polyelectrolyte ion exchange membrane, acrylic copolymer/nylon, and polyethersulfone. In a preferred embodiment, the membrane is made of cellulose nitrate (e.g., a cellulose nitrate membrane with a Mylar backing). The lateral flow solid phase apparatus can also optionally include other features, including sample pads, wicking pads, internal standard components, control components (e.g., a control zone), or other features.

The capillary flow solid phase apparatus can be any solid phase apparatus designed for a capillary flow assay, such as the BioSite TRIAGE immunoassay products (BioSite Inc., San Diego, Calif.). Generally, the capillary flow solid phase apparatus includes a capillary channel through which the test sample will flow.

Whether a lateral flow solid phase apparatus or a capillary flow solid phase apparatus is used, the solid phase apparatus has an application point and one or more sample capture zones. Generally, one sample capture zone is used, although more than one can be used, for example, when two slightly different target analytes are to be assessed simultaneously. The application point is the position on the membrane or in the capillary channel where the test sample can be applied. A sample capture zone refers to a point on the membrane or in the capillary channel at which at least one sample capture reagent is adsorbed (e.g., coated on and/or permeated through the membrane, or coated on the surface of the capillary channel). As used herein, the term “adsorbed” indicates that the agent is immobilized or adhered by non-covalent interactions, in contrast to covalent linkage where chemical means are used to generate an irreversible chemical bond of shared electrons between two linked molecules. Incremental movement (e.g., desorbtion) of an agent that is adsorbed onto a membrane or a surface of a capillary channel may occur, but will have negligible affect on the assays of the invention.

A sample capture reagent is an analyte binding agent, such as those described herein. A sample capture reagent need not be the same analyte binding agent as described in relation to analyte binding agents on the detection particles; however, each sample capture reagent also forms a binding pair with the target analyte, in that it specifically and preferentially binds to the target analyte. More than one sample capture reagent can be present at the sample capture zone, if desired.

In one embodiment, the detection particles can be positioned (e.g., dried or otherwise applied) onto the membrane of the lateral flow solid phase apparatus or the surface of the capillary flow solid phase apparatus (e.g., at or downstream from the application point). In this embodiment, they are releasably attached to the solid phase apparatus in such a manner that upon application of fluid to the solid phase apparatus, they can be moved by capillary action of the fluid. In another embodiment; the detection particles can be maintained in a separate sample collection apparatus that is utilized for collection of the test sample. Representative sample collection apparatus include a sample tube, a test tube, a vial; a pipette or pipette tip, or a syringe. If the detection particles are contained within the sample collection apparatus are stored in a stable form within the sample collection apparatus. A “stable form,” as the term is used herein, indicates a form in which the particles do not significantly change in chemical makeup or physical state during storage. The stable form can be a liquid, gel, or solid form. In preferred embodiments, the detection particles contained within a sample collection apparatus are evaporatively dried; freeze-dried; and/or vacuum-dried.

To perform the assay, a test sample to be assessed for the presence of a target analyte, as described above, is used. If the detection particles are on the solid phase apparatus, the test sample can be directly applied to the solid phase apparatus (e.g., at the application point); alternatively, it can be diluted or otherwise exposed to a buffer or other ingredients prior to application to the solid phase apparatus. In the embodiment in which the detection particles are maintained in a separate sample collection apparatus, the test sample is introduced into (drawn into, poured into, or otherwise placed into) the sample collection apparatus. For example; in one embodiment, the test sample is drawn up into a sample collection apparatus that comprises a pipette tip. Introduction of the test sample into the sample collection apparatus results in mixing of the test sample with the detection particles. If the detection particles are evaporatively-, freeze- or vacuum-dried, the introduction of the test sample into the sample collection apparatus can result in rehydration and suspension of the detection particles in the test sample. The test sample comprising rehydrated, suspended detection particles is then applied to the solid phase apparatus. Regardless of whether the detection particles are on the solid phase apparatus or in a separate sample collection apparatus, once the test sample comes into contact with the detection particles; binding of the target analyte (if present) in the test sample to the detection particles commences.

Once the test sample has been applied to the solid phase apparatus, the solid phase apparatus is then subjected to elevated heat. “Elevated” heat, as used herein, indicates that the temperature is at least about 5 degrees Celsius higher than ambient temperature (e.g., unheated or uncontrolled temperature, such as room temperature, which it typically approximately 21 degrees Celsius). In representative embodiments, the temperature is at least about 5 to 20 degrees Celsius higher than ambient temperature, such as at about between 35 to 40 degrees Celsius, inclusive (e.g., 37 degrees Celsius).

Generally, the solid phase apparatus can be heated to a temperature greater than about 25° C. and less than about 45° C. In some embodiments, the solid phase apparatus can be heated to a temperature of at least 25° C. such as, for example, at least 27° C., at least 29° C., at least 37° C., or at least 42° C. In some embodiments, the solid phase apparatus can be heated to a temperature of no more than 45° C. such as, for example, no more than 42° C., no more than 37° C., no more than 29° C., or no more than 27° C.

In other embodiments, the solid phase apparatus may be incubated at a desired temperature with limited temperature variability during the incubation period. In some embodiments, the solid phase apparatus may be incubated so that the temperature varies during incubation by no more than ±1° C. such as, for example, so that temperature varies by no more than 0.5° C. or so that the temperature varies by no more than 0.1° C.

Thus, in some embodiments, the method can provide reduced non-specific binding between the target analyte, if present in the sample, and a capture reagent in a solid phase apparatus compared to incubating the sample and solid phase apparatus at ambient temperature.

In other embodiments, the method can provide increased specific binding between the target analyte, if present in the sample, and a capture reagent in a solid phase apparatus compared to incubating the sample and solid phase apparatus at ambient temperature.

In still other embodiments, the method can increase the ratio of specific to non-specific binding between the target analyte, if present in the sample, and a capture reagent in a solid phase apparatus compared to incubating the sample and solid phase apparatus at ambient temperature.

While the solid phase apparatus is being subjected to elevated heat, the lateral flow or capillary flow assay progresses. For example if the target analyte is present in the test sample, continued binding occurs between the target analyte and the detection particles and continues as the assay progresses. “Binding” of the target analyte to detection particles indicates that an analyte binding agent coated onto the particle is interacting with (e.g., binding to) the target analyte. The detection particles may or may not have the target analyte bound to the analyte binding agent, depending on whether or not the target analyte is present in the fluid sample and whether the target analyte has bound to the analyte binding agent on the detection particles. Thus, a population of detection particles may comprise particles having various amount of target analyte bound to the analyte binding agents, as well as particles having no target analyte bound to the analyte binding agents (just as the detection particles initially have no target analyte bound to the analyte binding agent).

Fluid moves by capillary action to and through the solid phase apparatus. The detection particles, as well as target analyte, if present in the test sample, move through the solid phase apparatus as a result of capillary action of the fluid, and the detection particles move along the membrane (or along the capillary) to and through the sample capture zone(s). The movement of some of the detection particles is arrested by binding of detection particles to sample capture reagent(s) in the sample capture zone. Sample capture reagent binds to contacted detection particles by binding to target analyte which is bound to analyte binding agent on the contacted detection particles.

To assess the amount of detection particles arrested in a sample capture zone, any appropriate means for the type of label used on the detection particles can be used. For example, the amount can be detected by an optical method, such as by measuring the amount of fluorescence of the label of the detection particles. Alternatively; the amount of particles can be detected using electrical conductivity or dielectric (capacitance). Alternatively, electrochemical detection of released electroactive agents, such as indium, bismuth, gallium or tellurium ions can be used. For example; if liposomes are used, ferrocyanide encapsulated within the liposome can be released by addition of a drop of detergent at the capture zone, and the released ferrocyanide detected electrochemically. If chelating agent-protein conjugates are used to chelate metal ions, addition of a drop of acid at the capture zone will release the ions and allow quantitation by anodic stripping voltametry.

The elevated temperature to which the solid phase apparatus is exposed results in a decrease of non-specific binding of detection particles in the sample capture zone. “Non-specific binding,” as used herein refers to the arrest of detection particles in a sample capture zone. This is due to binding of the detection particles despite an absence of target analyte. Non-specific binding is distinct from “background” binding, which occurs throughout the membrane (in lateral flow solid phase assays) or throughout the capillary tube (in capillary flow solid phase assays). Non-specific binding is in excess of the expected or measured background binding. A “decrease” in non-specific binding, as used herein, refers to an amount of non-specific binding that is significantly less than the amount of binding that would occur in the absence of elevated temperature. An amount is significantly less, for example, if the mean value for binding in the presence of elevated temperature differs by at least 10% from the mean value for binding in the absence of elevated temperature. In other embodiments, there is a difference of at least one standard deviation between the mean value for binding in the presence of elevated temperature and the mean value for binding in the absence of elevated temperature.

The methods of the invention enhance the sensitivity and specificity of lateral flow and capillary flow assays for target analytes. Lower non-specific binding results in enhanced sensitivity in the assay, because it allows easier resolution of low concentrations of specific binding. Enhancement of specific binding similarly allows for easier resolution of low concentrations of specific binding, thereby enabling detection of the presence of smaller quantities of target analyte in a test sample. The accuracy of test results, over a broader concentration range for the analyte, is thereby increased.

Analyzing for the presence or absence of the specific microorganism (e.g., a bacterium) can result from detecting the presence of one or more target analytes or the absence of all analytes.

Thus, in some embodiments, the method can include: providing a sample suspected of including a target analyte characteristic of a microorganism; providing at least one labeled analyte binding agent having specificity for at least one target analyte (e.g., a microorganism); providing contact between the sample and the at least one labeled analyte binding agent; providing contact between the sample and a sample capture zone comprising at least one capture reagent, wherein the capture reagent possesses specificity for the at least one target analyte; incubating the solid phase apparatus at a temperature greater than about 25° C. and less than about 45° C.; and detecting label in the sample capture zone, wherein detecting label over background indicates the presence of the target analyte in the sample.

Generally, the solid phase apparatus is heated after the sample is applied to the application point and before the bound target analyte is detected. As noted previously, unless otherwise indicated in a particular embodiment, the sample may be applied to the solid phase apparatus and the solid phase apparatus heated prior to, simultaneously with, or subsequent to (or an appropriate combination thereof) applying the sample with the labeled analyte binding agent. For example, the sample may be applied to the solid phase apparatus and heated prior to contacting the sample with the labeled analyte binding agent. In another example, the sample may be simultaneously contacted with the sample capture zone and the labeled analyte binding agent, and the solid phase apparatus subsequently heated. In another example, the sample may be first contacted with the labeled analyte binding agent, then contacted with the sample capture zone, and the solid phase apparatus subsequently heated. Other variations are readily apparent to those skilled in the art.

In one such embodiment, the method further includes providing a solid phase apparatus that comprises the sample capture zone; wherein the sample and the analyte binding agent are contacted with one another prior to contacting the sample and the sample capture zone.

In other embodiments, the method can include: providing a sample suspected of including a target analyte characteristic of a microorganism; providing an solid phase apparatus comprising: an application point comprising at least one labeled analyte binding agent having specificity for at least one target analyte of the microorganism, and a sample capture zone comprising at least one capture reagent having specificity for the at least one target analyte; applying the sample to the application point of the solid phase apparatus, thereby providing contact between target analyte, if present in the sample, and the labeled analyte binding agent and generating labeled analyte; providing contact between the sample and the sample capture zone; incubating the solid phase apparatus at a temperature greater than about 25° C. and less than about 45° C.; and detecting labeled analyte in the sample capture zone, wherein detecting label over background indicates the presence of the target analyte in the sample.

In one particular embodiment, providing contact between the sample, the labeled analyte binding agent, and the sample capture zone of an solid phase apparatus can include: contacting the sample with a labeled analyte binding agent (this contact may occur, for example, within a contact region on the solid phase apparatus or before the sample enters the solid phase apparatus); and contacting the labeled analyte binding agent/sample with the sample capture zone of the solid phase apparatus.

In another particular embodiment, providing contact between the sample, the labeled analyte binding agent, and the sample capture zone of a solid phase apparatus can include: placing the sample into the solid phase apparatus; and subsequently contacting the sample in the device with the labeled analyte binding agent and the capture reagent.

In yet another embodiment, the method can include contacting the sample and the labeled analyte binding agent with unlabeled ligand corresponding to the target analyte such that the unlabeled ligand can bind specifically to the at least one capture reagent and, therefore, compete with the target analyte for binding sites within the sample capture zone.

Method of Detecting S. aureus:

The invention will now be described with regard to the detection of a specific target analyte—Staphylococcus aureus. The present invention pertains to methods for decreasing non-specific binding in solid phase assays for the assessment of Staphylococcus aureus. The solid phase assays of the invention are lateral flow solid phase assays or capillary flow solid phase assays.

In these embodiments, an assay refers to an in vitro procedure for analysis of a sample to determine the presence, absence, or quantity of the analyte of interest, Staphylococcus aureus. The methods of the invention utilize analyte binding agents that are members of a specific binding pair, in which a first member of the binding pair (the analyte—Staphylococcus aureus) reacts specifically with a second member (e.g., an analyte binding agent). Specific interaction between the members of the binding pair indicates that the first member of the binding pair preferentially binds or otherwise interacts with the second member of the binding pair, preferably to the exclusion of any binding to another compound in the assay. In one embodiment, the second member of the binding pair can be an antibody to Staphylococcus aureus (e.g., antibody to an antigenic component of Staphylococcus aureus). In this embodiment, the assay is an immunoassay. In another embodiment, the second member of the binding pair can be fibrinogen. The “analyte of interest,” as used in these embodiments, refers to Staphylococcus aureus, and can be an epitope, antigen or component (e.g.; a protein) of Staphylococcus aureus, as well as the entire Staphylococcus aureus organism.

In the methods of these embodiments, a test sample is assessed. The test sample can either be a sample which is suspected of containing Staphylococcus aureus, or a test sample which is not suspected of containing Staphylococcus aureus. In one embodiment: the test sample is a mucosal sample. A mucosal sample refers to samples that include, or are derived from, mucosal membranes and mucosal tissues, which are used interchangeably and refer to the surfaces of the nasal (including anterior nares, nasopharyngeal cavity, etc.), oral (e.g., mouth), outer ear, middle ear, vaginal cavities, and other similar tissues. Examples include samples including or derived from mucosal membranes such as buccal, gingival, nasal, ocular; tracheal, bronchial, gastrointestinal, rectal, urethral, ureteral; vaginal, cervical, and uterine mucosal membranes. In another embodiment, the test sample is a sample that includes, or is derived from, another bodily fluid (e.g., blood, saliva, cerebrospinal fluid, pus) or includes or is derived from a scraping or swab of a bodily tissue (e.g.; a skin scraping). If desired, the test sample can be diluted or otherwise treated; for example, it can be diluted with a solution (e.g., an aqueous solution or buffer), or subjected to a mucolytic agent.

The assays utilize “staph detection particles.” The staph detection particles are particles which can be coated with an analyte binding agent (the second member of the binding pair) for Staphylococcus aureus. In a preferred embodiment, the staph detection particles are liposomes, colloidal gold; organic polymer latex particles, inorganic fluorescent particles or phosphorescent particles. In a particularly preferred embodiment, the particles are polystyrene latex beads, and most particularly, polystyrene latex beads that have been prepared in the absence of surfactant, such as surfactant free Superactive Uniform Aldehyde/Sulfate Latexes (Invitrogen Corp.; Carlsbad, Calif.). Generally, a single population of staph detection particles is used; the staph detection particles are coated with at least one (one or more) type(s) of analyte binding agent for Staphylococcus aureus. For example; a single population of staph detection particles, coated with one or more types of analyte binding agent, or more than one population of staph detection particles, each population coated with one or more types of analyte binding agent, can be used. Representative analyte binding agents include antibodies (or fragments thereof); haptens; drug conjugates; receptors; or other binding partners. In one preferred embodiment, an analyte binding agent is an antibody to Staphylococcus aureus or to an antigen of Staphylococcus aureus, such as an antibody to protein A or to clumping factor of Staphylococcus aureus; in another preferred embodiment, an analyte binding agent is fibrinogen.

Antibodies can be monoclonal antibodies or polyclonal antibodies. The term “antibody”, as used in these embodiments, also refers to antibody fragments which are sufficient to bind to Staphylococcus aureus (e.g., to an antigen of Staphylococcus aureus). Alternatively: in another embodiment, molecules which specifically bind to Staphylococcus aureus, such as engineered proteins having Staphylococcus aureus or Staphylococcus aureus antigen binding sites, can also be used (Holliger, P. and H. R. Hoogenbloom, Trends in Biotechnology 13:7 9 (1995); Chamow, S. M. and A. Ashkenazi, Trends in Biotechnology 14:52 60:1996)).

The staph detection particles must be sufficiently small to be transported along a membrane or through a capillary channel by capillary action of fluid, and also sufficiently small for a complex of Staphylococcus aureus and staph detection particles, as described below, to be transported by capillary action. The particles can be labeled to facilitate detection. The particles are labeled by a means which does not significantly affect the physical properties of the particles; for example, the particles are labeled internally (that is; the label is included within the particle, such as within the liposome or inside the polystyrene latex bead). Representative labels include luminescent labels; chemiluminescent labels; phosphorescent labels; enzyme-linked labels; chemical labels, such as electroactive agents (e.g., ferrocyanide); and colorimetric labels, such as dyes or fluorescent labels. In one embodiment, a fluorescent label is used. In another embodiment, phosphorescent particles are used, particularly “up-converting” phosphorescent particles, such as those described in U.S. Pat. No. 5,043,265.

The assays of the invention also utilize a solid phase apparatus. In one embodiment, the solid phase apparatus is a lateral flow solid phase apparatus. In the other embodiment, the solid phase apparatus is a capillary flow solid phase apparatus.

The lateral flow solid phase apparatus can be any solid phase apparatus designed for a lateral flow assay, such as the RAMP™ apparatus (Response Biomedical, Burnaby, British Columbia, Canada; see, e.g., apparatus described in U.S. Pat. Nos. 6,509,196; 7,175,992). Generally, the lateral flow solid phase apparatus includes a membrane through which the test sample will flow. The membrane can be made of a substance having the following characteristics: sufficient porosity to allow capillary action of fluid along its surface and through its interior; the ability to allow movement of coated particles (e.g., staph detection particles as described herein) or complexes of particles and Staphylococcus aureus or Staphylococcus aureus components, by capillary action (i.e., it must not block the particles or complexes of particles and analyte of interest); and the ability to be wet by the fluid containing the analyte (e.g., hydrophilicity for aqueous fluids, hydrophobicity for organic solvents). Hydrophobicity of a membrane can be altered to render the membrane hydrophilic for use with aqueous fluid, by processes such as those described in U.S. Pat. No. 4,340,482 or U.S. Pat. No. 4,618,533, which describe transformation of a hydrophobic surface into a hydrophilic surface. Examples of membrane substances include: cellulose, cellulose nitrate, cellulose acetate, glass fiber, nylon, polyelectrolyte ion exchange membrane, acrylic copolymer/nylon, and polyethersulfone. In a preferred embodiment, the membrane is made of cellulose nitrate (e.g., a cellulose nitrate membrane with a Mylar backing). The lateral flow solid phase apparatus can also optionally include other features, including sample pads, wicking pads, internal standard components, control components, or other features.

The capillary flow solid phase apparatus can be any solid phase apparatus designed for a capillary flow assay, such as the BioSite TRIAGE immunoassay products (BioSite Inc., San Diego, Calif.). Generally, the capillary flow solid phase apparatus includes a capillary channel through which the test sample will flow.

Whether a lateral flow solid phase apparatus or a capillary flow solid phase apparatus is used in these embodiments, the solid phase apparatus has an application point and one or more sample capture zones. Generally, one sample capture zone is used, although more than one can be used, for example, when two slightly different types of staph are to be assessed (e.g., ordinary staph and drug-resistant staph). The application point is the position on the membrane or in the capillary channel where the test sample can be applied. A sample capture zone refers to a point on the membrane or in the capillary channel at which at least one (one or more) sample capture reagent(s) is adsorbed (e.g., coated on and/or permeated through the membrane, or coated on the surface of the capillary channel). As used herein, the term “adsorbed” indicates that the agent is immobilized or adhered by non-covalent interactions, in contrast to covalent linkage where chemical means are used to generate an irreversible chemical bond of shared electrons between two linked molecules. Incremental movement (e.g., desorbtion) of an agent that is adsorbed onto a membrane or a surface of a capillary channel may occur, but will have negligible affect on the assays of the invention.

In these embodiments, a sample capture reagent is an analyte binding agent, such as those described above. A sample capture reagent need not be the same analyte binding agent as described in relation to analyte binding agents on staph detection particles; however, each sample capture reagent also forms a binding pair with Staphylococcus aureus (or a component of Staphylococcus aureus), in that it specifically and preferentially binds to Staphylococcus aureus. In a preferred embodiment, a sample capture reagent is an antibody directed against Staphylococcus aureus (e.g., against an epitope; antigen or component of Staphylococcus aureus); for example, it can be directed against the same epitope, antigen or component of Staphylococcus aureus as, or against a different epitope, antigen or component of the analyte from, the epitope, antigen or component that binds to the antibodies used as analyte binding agents coated on the staph detection particles. More than one sample capture reagent can be present at the sample capture zone: if desired. For example, antibody to protein A of Staphylococcus aureus and antibody to Clumping Factor of Staphylococcus aureus can be used as sample capture reagents, either alone or in combination.

In one embodiment, the staph detection particles can be positioned (e.g., dried or otherwise applied) onto the membrane of the lateral flow solid phase apparatus or the surface of the capillary flow solid phase apparatus (e.g., at or downstream from the application point). In this embodiment, they are attached to the solid phase apparatus in such a manner that upon application of fluid to the solid phase apparatus, they can be moved by capillary action of the fluid. In another embodiment; the staph detection particles can be maintained in a separate sample collection apparatus that is utilized for collection of the test sample. Representative sample collection apparatus include a sample tube, a test tube, a vial; a pipette or pipette tip, or a syringe. If the staph detection particles are contained within the sample collection apparatus are stored in a stable form within the sample collection apparatus. A “stable form,” as the term is used herein, indicates a form in which the particles do not significantly change in chemical makeup or physical state during storage. The stable form can be a liquid, gel, or solid form. In preferred embodiments, the staph detection particles contained within a sample collection apparatus are evaporatively dried; freeze-dried; and/or vacuum-dried.

To perform the assay, a test sample to be assessed for the presence of Staphylococcus aureus, as described above, is used. If the staph detection particles are on the solid phase apparatus, the test sample can be directly applied to the solid phase apparatus (e.g., at the application point); alternatively, it can be diluted or otherwise exposed to a buffer or other ingredients prior to application to the solid phase apparatus. In the embodiment in which the staph detection particles are maintained in a separate sample collection apparatus, the test sample is introduced into (drawn into, poured into, or otherwise placed into) the sample collection apparatus. For example; in one embodiment, the test sample is drawn up into a sample collection apparatus that comprises a pipette tip. Introduction of the test sample into the sample collection apparatus results in mixing of the test sample with the staph detection particles, so that the test sample now comprises the particles. If the staph detection particles are evaporatively-, freeze- or vacuum-dried, the introduction of the test sample into the sample collection apparatus can result in rehydration and suspension of the staph detection particles in the test sample. The test sample comprising rehydrated, suspended staph detection particles is then applied to the solid phase apparatus. Regardless of whether the staph detection particles are on the solid phase apparatus or in a separate sample collection apparatus, once the test sample comes into contact with the staph detection particles; binding of Staphylococcus aureus (if present) in the test sample to the staph detection particles commences.

Once the test sample has been applied to the solid phase apparatus, the solid phase apparatus is then subjected to elevated heat. “Elevated” heat, as used in these embodiments, indicates that the temperature is at least about 5 degrees Celsius higher than ambient temperature (e.g., unheated or uncontrolled temperature. such as room temperature, which it typically approximately 21 degrees Celsius). In representative embodiments, the temperature is at least about 5 to 20 degrees Celsius higher than ambient temperature, such as at about between 35 to 40 degrees Celsius, inclusive (e.g., 37 degrees Celsius).

While the solid phase apparatus is being subjected to elevated heat, the lateral flow or capillary flow assay progresses. For example if Staphylococcus aureus is present in the test sample, continued binding occurs between the Staphylococcus aureus and the staph detection particles and continues as the assay progresses. “Binding” of Staphylococcus aureus to staph detection particles indicates that an analyte binding agent coated onto the particle is interacting with (e.g., binding to) its analyte of interest (Staphylococcus aureus). Staph detection particles may or may not have Staphylococcus aureus bound to the analyte binding agent, depending on whether or not Staphylococcus aureus is present in the fluid sample and whether Staphylococcus aureus has bound to the analyte binding agent on the staph detection particles. Thus, a population of staph detection particles may comprise particles having various amount of Staphylococcus aureus bound to the analyte binding agents, as well as particles having no Staphylococcus aureus bound to the analyte binding agents (just as the staph detection particles initially have no Staphylococcus aureus bound to the analyte binding agent).

Fluid moves by capillary action to and through the solid phase apparatus. Staph detection particles, as well as Staphylococcus aureus if present in the test sample, move through the solid phase apparatus as a result of capillary action of the fluid, and the staph detection particles move along the membrane (or along the capillary) to and through the sample capture zone(s). The movement of some of the staph detection particles is arrested by binding of staph detection particles to sample capture reagent(s) in the sample capture zone. Sample capture reagent binds to contacted staph detection particles by binding to Staphylococcus aureus which is bound to analyte binding agent on the contacted staph detection particles.

To assess the amount of staph detection particles arrested in a sample capture zone, any appropriate means for the type of label used on the staph detection particles can be used. For example, the amount can be detected by an optical method, such as by measuring the amount of fluorescence of the label of the staph detection particles. Alternatively; the amount of particles can be detected using electrical conductivity or dielectric (capacitance). Alternatively, electrochemical detection of released electroactive agents, such as indium, bismuth, gallium or tellurium ions, as described by Hayes et al, (Analytical Chem. 66:1860-1865 (1994)) or ferrocyanide as suggested by Roberts and Durst (Analytical Chem. 67:482-491 (1995)) can be used. For example; if liposomes are used, ferrocyanide encapsulated within the liposome can be released by addition of a drop of detergent at the capture zone, and the released ferrocyanide detected electrochemically (Roberts and Durst, id.). If chelating agent-protein conjugates are used to chelate metal ions, addition of a drop of acid at the capture zone will release the ions and allow quantitation by anodic stripping voltametry (Hayes et al., id.).

The elevated temperature to which the solid phase apparatus is exposed results in a decrease of non-specific binding of staph detection particles in the sample capture zone. “Non-specific binding,” as used in these embodiments refers to the arrest of staph detection particles in a sample capture zone. This is due to binding of the staph detection particles despite an absence of Staphylococcus aureus. Non-specific binding is distinct from “background” binding, which occurs throughout the membrane (in lateral flow solid phase assays) or throughout the capillary tube (in capillary flow solid phase assays). Non-specific binding is in excess of the expected or measured background binding. A “decrease” in non-specific binding, as used herein, refers to an amount of non-specific binding that is significantly less than the amount of binding that would occur in the absence of elevated temperature. An amount is significantly less, for example, if the mean value for binding in the presence of elevated temperature differs by at least 10% from the mean value for binding in the absence of elevated temperature. In other embodiments, there is a difference of at least one standard deviation between the mean value for binding in the presence of elevated temperature and the mean value for binding in the absence of elevated temperature.

The methods of these embodiments of the invention enhance the sensitivity and specificity of lateral flow and capillary flow assays for Staphylococcus aureus. Lower non-specific binding results in enhanced sensitivity in the assay, because it allows easier resolution of low concentrations of specific binding. Enhancement of specific binding similarly allows for easier resolution of low concentrations of specific binding, thereby enabling detection of the presence of smaller quantities of Staphylococcus aureus in a test sample. The accuracy of test results is increased and enables more accurate treatment of disease.

The invention is now illustrated by the following Exemplification, which is not intended to be limiting in any way.

The invention is now illustrated by the following Exemplification, which is not intended to be limiting in any way.

### Exemplification

### Reduction of Non-Specific Binding and Increase of Specific Binding in Staphylococcus aureus Assays

Lateral flow immunoassay solid phase apparatus was prepared using a nitrocellulose membrane, with two antibody specificities for protein A and Clumping Factor obtained from 3M manufactured at Strategic Biosolutions™, one at 1.4 mg/ml and one at 0.9 mg/mL, in 10 mM Phosphate Buffer with Azide, pH 7.2, filtered 1 μl/cm Staphylococcus aureus antibody (sample capture reagent) striped at TL-position (sample capture zone). and 0.75 mg/ml each of 1 μl/cm Goat anti-mouse antibody (Arista Biologicals Inc.) and Goat anti-rabbit antibody (CedarLane Laboratories Ltd, manufacturer Jackson Immunology Research Laboratories Ltd.) (control capture reagent) striped at ISL-position (control capture zone). The antibodies were striped on nitrocellulose membrane (with or without pre-washing, as needed) which was then blocked with at most 1% PVA, washed with 2 mM Phosphate Buffer solution as needed, and then dried and cut into 5 mm or less wide strips.

RAMP™ test cartridges were assembled using the strips, a sample pad, and a wicking pad.

Latex-antibody conjugate (staph detection particles) were prepared as follows: 0.25 mg of two different Staphylococcus aureus antibodies (MAb-76 anti-protein A & polyclonal anti-Clumping Factor and 0.5 g of fluorescent dyed latex beads in 10 mM PB solution were allowed to non-covalently adsorb by incubating at 2-8° C. for 12-18 hours. Cyanoborohydride in 1% Skim milk was added and incubated at 2-8° C. for 2-3 hours to form the covalent bonds of the latex-antibody conjugate and block non-specific binding. The latex-antibody conjugate was then washed several times using 10 mM PB solution to remove the cyanoborohydride and skim milk solution.

The latex-antibody conjugate was spotted on to a cap or a pipette tip and dried using a vacuum pump or convection oven to prepare RAMP™ assay caps or tips (sample collection apparatus).

Sample Buffer was prepared using 138 mM PB: 138 mM NaCl, 3.56% BSA, 0.82% Surfactant 10 G. 0.592% casein, 0.6% PVP-k90, 0.1% v/v ProClin 300, 0.1% v/v ProClin 950 pH 7.2.

### B. Methods

A test sample was obtained by taking a swab and sampling the anterior nares with two rotations per nostril, and then was diluted by adding the sample directly into liquid sample buffer. The sample was then treated to release the antigen and remove interfering matrices (1.2% N-acetyl cysteine & 0.2% Sodium Dodecyl Sulfate). The staph detection particles were added to the prepared sample by mixing the sample using the pipette tip or by adding the latex containing cap to the sample vial and inverting and vortexing the sample. The sample was then added into the RAMP™ test cartridge and cartridge was inserted in the RAMP™ Fluorescence Reader (Response Biomedical), which was modified to have a heating block, so that placing the cartridge over the heating block resulted in the temperature inside the cartridge of 37° C. The assay was incubated at ambient temperature (23° C.), at 29° C., or at 37° C. for the duration of the assay development time. After 20 minutes heated development time, the cartridge was scanned using the RAMP™ Fluorescence Reader. Fluorescence measurements were measured at the TL, ISL, and corresponding background positions of the strip. The TL and ISL signals were corrected by subtracting the corresponding background signals. Calculation of ratios for the assays were performed by the reader as follows:

Staph Ratio=R10=TL/(TL+ISL)

Use of the ratio reduces the variability and increases the accuracy of the results, as compared to using only the TL, signals due to inherent variability of the solid phase apparatus and sample-to-sample variability. These variabilities affect the TL and ISL signals similarly and thus the ratio value is used to reduce the variability in test results between multiple samples run.

### C. Results

Increased Sensitivity (Decreasing Non-Specific Binding): The experiment showed that when the assay was performed with heating to achieve an internal cartridge temperature of 37° C., the sensitivity of the assay for detection of Staphylococcus aureus antigen was increased compared to the same assay performed at ambient temperature (approx. 25° C.) or at 29° C. The non-specific binding at the zero sample decreased from TL signal of 65+/−6 F-units @˜25° C. to 44+/−3 F-units @ 37° C., a 32% decrease with no overlap in error bars; the specific binding stayed similar at 8.7×10⁴ CFU/mL: a high positive, with TL signal of 143+/−7 F-units @−25° C. to 133+/−4 F-units with overlapping error bars which increased the distance between the negative and the high positive sample. This resulted in an increase of the ratio of TL Specific to Non-Specific (Sp:NonSp) from 2.21 @−25° C. to 3.04 @ 37° C. This increase in distance between the negative and positive and the increase in ratio of TL Specific to Non-specific correlates with an increase in sensitivity in the Staph A assay at the higher temperature of 37° C. relative to 25° C.

TABLE 1
Results of assessment of specificity in heated vs. unheated staph assays
TL

| Temperature | M73 (cfu/ml) | mean | sd | % cv | Sp: nonsp | Sp-nonsp |
|---|---|---|---|---|---|---|
| 25° C. | 0.00^{E}+00 | 65 | 6 | 8.6 | — | — |
| | 8.70E+04 | 143 | 7 | 5.0 | 2.21 | 66 |
| 29° C. | 0.00E+00 | 55 | 5 | 9.0 | — | — |
| | 8.70E+04 | 139 | 10 | 7.3 | 2.53 | 69 |
| 37° C. | 0.00E+00 | 44 | 3 | 5.9 | — | — |
| | 8.70E+04 | 133 | 4 | 3.2 | 3.04 | 82 |

ISL

| Temperature | M73 (cfu/ml) | mean | sd | % cv |
|---|---|---|---|---|
| 25° C. | 0.00^{E}+00 | 4435 | 345 | 7.8 |
| | 8.70E+04 | 4296 | 523 | 12.2 |
| 29° C. | 0.00E+00 | 4140 | 365 | 8.8 |
| | 8.70E+04 | 4346 | 394 | 9.1 |
| 37° C. | 0.00E+00 | 4219 | 201 | 4.8 |
| | 8.70E+04 | 4343 | 377 | 8.7 |

R10

| Temperature | M73 (cfu/ml) | mean | sd | % cv | Sp: nonsp | Sp-nonsp |
|---|---|---|---|---|---|---|
| 25° C. | 0.00E+00 | 0.0145 | 0.0017 | 11.8 | — | — |
| | 8.70E+04 | 0.0326 | 0.0047 | 14.5 | 2.25 | 0.0117 |
| 29° C. | 0.00E+00 | 0.0132 | 0.0013 | 9.7 | — | — |
| | 8.70E+04 | 0.0311 | 0.0021 | 6.7 | 2.37 | 0.0146 |
| 37° C. | 0.00E+00 | 0.0103 | 0.0009 | 9.1 | — | — |
| | 8.70E+04 | 0.0291 | 0.0027 | 9.4 | 2.83 | 0.0151 |

The FIGURE demonstrates the increased sensitivity of the lateral flow solid phase assay for staph at an elevated temperature, compared to an intermediate temperature and to ambient temperature. The signal demonstrates that elevated temperature consistently results in greater specific binding, yielding increased sensitivity in the assay.

The present invention has now been described with reference to several specific embodiments foreseen by the inventor for which enabling descriptions are available. Insubstantial modifications of the invention, including modifications not presently foreseen, may nonetheless constitute equivalents thereto. Thus, the scope of the present invention should not be limited by the details and structures described herein, but rather solely by the following claims, and equivalents thereto.

1. A method of analyzing a sample for a target analyte, the method comprising:
providing a fluid sample suspected of including a target analyte;
providing at least one labeled analyte binding agent having specificity for the target analyte;
providing a solid phase apparatus comprising:
a sample application point, and
a sample capture zone comprising at least one sample capture reagent having specificity for the target analyte;
providing contact between the fluid sample and the labeled analyte binding agent whereby the target analyte, if present in the sample, becomes a labeled analyte;
applying the fluid sample to the application point whereby the sample moves by capillary action through the solid phase apparatus to and through the sample capture zone;
incubating the solid phase apparatus at a temperature greater than about 25° C. and less than about 45° C.; and
detecting labeled analyte in the sample capture zone, wherein detecting labeled analyte indicates the presence of the target analyte in the sample.

2. The method of claim 1, wherein the labeled analyte binding agent comprises particles.

3. The method of claim 2, wherein the particles are labeled particles.

4. The method of claim 2, wherein the particles are coated with at least one type of analyte binding agent that binds to the target analyte.

5. The method of claim 2, wherein the sample application point comprises the particles.

6. The method of claim 5, wherein the particles move to and through the sample capture zone, and wherein particles may bind to a sample capture reagent.

7. The method of claim 2; wherein the test sample comprises the particles, and wherein the application of the test sample to the solid phase apparatus applies the particles to the solid phase apparatus.

8. The method of claim 7, wherein the particles move to and through the sample capture zone, and wherein particles may bind to a sample capture reagent.

9. The method of claim 1, wherein the solid phase apparatus is a lateral flow solid phase apparatus or a capillary flow solid phase apparatus.

10. The method of claim 1, wherein the at least one labeled analyte binding agent comprises two or more analyte binding agents.

11. The method of claim 10 wherein the two or more analyte binding agents have specificity for at least two different target analytes.

12. The method of claim 1, wherein incubating the solid phase apparatus comprises incubating at a temperature greater than about 29° C.

13. The method of claim 1, wherein incubating the solid phase apparatus comprises incubating at a temperature greater than about 37° C.

14. The method of claim 1, wherein incubating the solid phase apparatus comprises incubating at a temperature less than about 42° C.

15. The method of claim 1, wherein incubating the solid phase apparatus comprises incubating at a temperature that varies during incubation by no more than ±1° C.

16. The method of claim 1, wherein incubating the solid phase apparatus comprises incubating at a temperature that varies during incubation by no more than ±0.5° C.

17. The method of claim 1, wherein incubating the solid phase apparatus comprises incubating at a temperature that varies during incubation by no more than ±0.1° C.

18. The method of claim 1 wherein incubating the solid phase apparatus comprises incubating at a temperature greater than about 25° C. and less than about 45° C. reduces non-specific binding between the target analyte, if present in the sample, compared to incubating the solid phase apparatus at ambient temperature.

19. The method of claim 1 wherein incubating the solid phase apparatus comprises incubating at a temperature greater than about 25° C. and less than about 45° C. increases specific binding between the target analyte, if present in the sample, compared to incubating the solid phase apparatus at ambient temperature.

20. The method of claim 1, wherein incubating the solid phase apparatus comprises incubating at a temperature greater than about 25° C. and less than about 45° C. increases the ratio of specific to non-specific binding between the target analyte and sample capture zone, if present in the sample, compared to incubating the solid phase apparatus at ambient temperature.

21. The method of claim 1, wherein at least one labeled analyte binding agent is a monoclonal antibody, a polyclonal antibody composition, fragments thereof, or a combination thereof.

22. The method of claim 21, wherein the at least one labeled analyte binding agent comprises two or more labeled antibodies.

23. The method of claim 22, wherein the two or more labeled antibodies comprises at least two different monoclonal antibodies.

24. The method of claim 1, wherein the sample capture zone comprises at least one immobilized antibody having antigenic specificity for the target analyte.

25. The method of claim 24, wherein the at least one immobilized antibody comprises two or more immobilized antibodies.

26. The method of claim 25, wherein the two or more immobilized antibodies are monoclonal antibodies, polyclonal antibodies, or a combination thereof.

27. The method of claim 25, wherein the two or more immobilized antibodies comprise at least two monoclonal antibodies with antigenic specificity for different epitopes.

28. The method of claim 21, wherein a labeled analyte binding agent or an antibody immobilized in the sample capture zone comprises an antibody raised against Protein A, Clumping Factor (Clf40), Type 5 capsular polysaccharide (CPS), or Type 8 CPS.

29. The method of claim 21, wherein a labeled analyte binding agent or an antibody immobilized in the sample capture zone comprises Mab-76 or Mab-107.

30. The method of claim 1, wherein at least one analyte binding agent comprises a lectin, a nucleic acid, or a cellular receptor.

31. The method of claim 1, wherein detecting the label comprises detecting a fluorophore.

32. The method of claim 31, wherein detecting a fluorophore comprises visually detecting a fluorophore without an instrument.

33. The method of claim 1, wherein the target analyte is characteristic of a microorganism selected from the group consisting of bacteria, yeast, filamentous fungi, and viruses.

34. The method of claim 33, wherein the microorganism comprises a virus.

35. The method of claim 34, wherein the virus comprises influenza virus.

36. The method of claim 33, wherein the microorganism is a bacterium.

37. The method of claim 36, wherein the bacterium comprises a species of Staphylococcus.

38. The method of claim 1, further comprising the step of treating the sample with a mucolytic agent.

39. The method of claim 1 wherein labeled analyte competes with a corresponding unlabeled ligand for the sample capture zone capture reagents.

40. The method of claim 1, wherein the step of providing contact between the sample and the analyte binding agent occurs prior to the step of providing contact between the sample and the sample capture zone.

41. A method for decreasing non-specific binding of staph detection particles on a solid phase apparatus in an assay for Staphylococcus aureus, the method comprising subjecting a solid phase apparatus having staph detection particles thereon to elevated temperature subsequent to application of a test sample to the solid phase apparatus, whereby the non-specific binding of the staph detection particles on the solid phase apparatus is decreased.

42. The method of claim 41, wherein the staph detection particles are coated with at least one type of agent that binds to Staphylococcus aureus.

43. The method of claim 42, wherein an agent that binds to Staphylococcus aureus is an antibody.

44. The method of claim 42, wherein an agent that binds to Staphylococcus aureus is fibrinogen.

45. The method of claim 42, wherein the solid phase apparatus comprises an application point and at least one sample capture zone having at least one sample capture reagent immobilized thereon.

46. The method of claim 45, wherein a sample capture reagent comprises an agent that specifically binds to Staphylococcus aureus.

47. The method of claim 45, wherein the staph detection particles are positioned at the application point and, upon application of the test sample, are moved by capillary action through the solid phase apparatus.

48. The method of claim 47, wherein the staph detection particles move to and through the sample capture zone, and wherein staph detection particles may bind to a sample capture reagent.

49. The method of claim 45, wherein the test sample comprises the staph detection particles, and wherein the application of the test sample to the solid phase apparatus applies the staph detection particles to the solid phase apparatus and results in movement of the staph detection particles though the solid phase apparatus by capillary action.

50. The method of claim 49, wherein the staph detection particles move to and through the sample capture zone, and wherein staph detection particles may bind to a sample capture reagent.

51. The method of claim 45, wherein the solid phase apparatus is a lateral flow solid phase apparatus.

52. The method of claim 45, wherein the solid phase apparatus is a capillary flow solid phase apparatus.

53. The method of claim 41, wherein the elevated temperature is about 5 to 20 degrees Celsius above ambient temperature.

54. The method of claim 53, wherein the elevated temperature is between about 35 and 40 degrees Celsius, inclusive.
